(19) United States
(12) Patent Application Publication
(10) Pub. No.: US 2017/0355940 A1
(43) Pub. Date: Dec. 14, 2017

(54) METHOD FOR VASCULARIZING IN-VITRO GENERATED OR EX-VIVO TISSUE FRAGMENTS IN A MICROFLUIDIC DEVICE

(71) Applicant: NORTIS, INC., Woodinville, WA (US)

(72) Inventors: Thomas Neumann, Seattle, WA (US); Mark E. Fauver, Seattle, WA (US); Richard Carleton Hulit, Seattle, WA (US)

(73) Assignee: NORTIS, INC., Woodinville, WA (US)

(21) Appl. No.: 15/527,222

(22) PCT Filed: Nov. 19, 2015
(86) PCT No.: PCT/US15/61642

§ 371 Date: May 16, 2017

Related U.S. Application Data
(60) Provisional application No. 62/082,110, filed on Nov. 19, 2014.

(51) Int. Cl. C12M 3/04; C12M 3/06; C12M 3/00; A61F 2/06

(52) U.S. Cl. CPC C12M 3/04; A61F 2/06; C12M 23/16; C12M 21/08

(57) ABSTRACT

Vascularizing cell aggregates or tissue segments in a microfluidic device by filling a chamber within the device with a matrix that allows for endothelial sprouting; creating at least three voids within the matrix, of which at least two outer voids are lumenally connected to separate perfusion paths within the device and at least one additional void is positioned in between the at least two outer voids; endothelializing the at least two outer voids; introducing at least one cell type, matrix material, tissue segment, or combinations thereof into the void between the two outer voids; and using vascular growth factors to induce the endothelial cells to sprout into the matrix until the at least three voids are interconnected by endothelial sprouts.

### CROSS-REFERENCE TO RELATED APPLICATIONS

This application is related to and claims the priority date of U.S. Provisional Application No. 62/082,110 to Neumann et al. entitled “METHOD FOR VASCULARIZING IN-VITRO GENERATED OR EX-VIVO TISSUE FRAGMENTS IN A MICROFLUIDIC DEVICE”, filed Nov. 19, 2014. U.S. Provisional Application No. 62/082,110 is incorporated by reference.

This application is also related to co-pending US PCT Application No. PCT/US2013/062307 to Tourovskaia et al. filed Sep. 27, 2013 and entitled “MICROFLUIDIC SYSTEM FOR REPRODUCING FUNCTIONAL UNITS OF TISSUES AND ORGANS IN VITRO,” the disclosure of which is incorporated by reference.

This application is also related to co-pending US PCT Application No. PCT/US2015/056271 to Neumann et al. filed Oct. 19, 2014 and entitled “MODULAR MICROFLUIDIC SYSTEM FOR PERFUSED CELL CULTURE,” the disclosure of which is incorporated by reference.

### FIELD OF THE INVENTION

The present invention relates to methods for reproducing functional units of vessel-like structures, and, more particularly, to systems including in-vitro or ex-vivo tissue vascularization.

### BACKGROUND OF THE INVENTION

The current literature teaches the creation of vessel-like structures in hydrogels. Known methods typically include the generation of voids within in these hydrogels. Voids can be generated through a number of methods, such as by mechanical extraction of mandrels, degradation of sacrificial structures within the hydrogel, or using soft lithography to generate stamps for molding channels into matrix materials, as published in the scientific literature. A second step includes seeding of endothelial cells or combinations of endothelial cells with other cells into these voids. This is usually done in microfluidic devices in which the voids within the hydrogel are in fluidic connection with perfusion channels in the chip.

Once these endothelialized vessel-like structures (e.g. parent vessels) are established they can be induced to sprout toward a gradient of vascular growth factors, such as VEGF. Inventors at Nortis, Inc., assignee of the present application, have previously demonstrated angiogenic sprouting from such parent vessels toward a gradient of VEGF/b-FGF/PMA (Tourovskaia et al. Exp Biol Med (Maywood), 2014, 239). That publication describes in detail the design of the microfluidic device as well as the cell sources and cell seeding protocols. Nortis inventors have also described the generation of microvascular networks derived from two parallel parent vessels that are induced to sprout in response to vascular growth factors present within the hydrogel matrix. The vascular networks are formed by anastomosis of branches from both parent vessels. Since each of these vessels is in independent fluidic connection with the fluidic channels in the microfluidic device, by creating a pressure difference within the parent vessels, fluid flow can be routed from one parent vessel through the anastomosed sprouts into the other parent vessel (U.S. Pat. No. 7,622,298 B2). Nortis inventors have also described the filling of empty voids with other cell types to create solid cords of tissue next to a sprouting parent vessel (See, for example, US PCT Application No. PCT/US2013/062307, also incorporated by reference).

Despite the aforesaid advances, there is an increased need for tissue-model systems that include a vascular and perfusion component for scientific research and drug development. For the first time, the present disclosure provides a solution comprising a method for generating a vascularized tissue component between two parent vessels within a microfluidic device in such a way that both parent vessels and the tissue component are interconnected by a network of capillaries. The models described in the present disclosure could serve as important tools to study a number of important diseases, such as cancer, cardiovascular disease, diabetes, inflammation, aging, and neurodegenerative diseases.

### SUMMARY OF THE DISCLOSURE

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Here disclosed is a method for vascularizing cell aggregates or tissue segments in a microfluidic device by filling a chamber within the device with a matrix that allows for endothelial sprouting; creating at least three voids within the matrix, of which at least two outer voids are lumenally connected to separate perfusion paths within the device and at least one additional void is positioned in between the at least two outer voids; endothelializing the at least two outer voids; introducing at least one cell type, matrix material, tissue segment, or combinations thereof into the void between the two outer voids; and using vascular growth factors to induce the endothelial cells to sprout into the matrix until the at least three voids are interconnected by endothelial sprouts.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawings, in which:

FIG. 1A schematically depicts an example of a 3-channel chip interior chamber, highlighting the fluidic channels and biological matrix chamber.

FIG. 1B schematically shows an example of using fluidic shutoff valves that enable rerouting of fluid through the mandrel channels.

FIG. 2A-FIG. 2C illustrate an example of a method of establishing two outer parent vessels and inducing them to sprout toward the center channel.

FIG. 2D-FIG. 2F demonstrate an example of the establishment of two quiescent parent vessels in the outer channels that are then induced to sprout toward the center channel by perfusion of growth factors through the center channel.

FIG. 3A1-FIG. 3C3 show vascularization of different tissue structures in a 3-channel chip. More specifically, FIG. 3A1-FIG. 3A2 illustrate a method for vascularizing multicellular structures; FIG. 3A3 shows connections between sprouts from outer parent HUVEC vessels and new microvessels and tumor cell clusters in the center channel; FIG. 3B1-FIG. 3B2 illustrate a method for creating a vascular network between angiogenic branches from outer parent vessels and vasculogenic microvessels formed from endothelial cells seeded with a gelable matrix in the center channel; FIG. 3B3 shows connections between sprouts from the outer parent HUVEC vessels and new microvessels formed by HUVEC cells embedded in collagen I and seeded into the central channel. CV, connecting vessels; FIG. 3C1 and FIG. 3C2 illustrate a method for vascularizing a central tubular tissue structure; and FIG. 3C3 shows sprouts from the outer parent HUVEC vessels attached to the ablumenal side of a central HUVEC vessel.

FIG. 4A-FIG. 4D jointly illustrate an example of a method for creating a vascularized pancreatic islet in a 3-channel chip.

FIG. 5A shows vascularization of tumor cell clusters in the center channel.

FIG. 5B and FIG. 5C demonstrate microvessels growing into and around tumor cell clusters.

FIG. 6 shows a series of video stills of fluorescent beads (10 um, arrows) traveling from one parent HUVEC vessel, through connecting capillaries, and out the second parent HUVEC vessel.

FIG. 7 illustrates an example of a 3 channel chamber and glue pockets thick layer.

FIG. 8 illustrates a detail from the 3 channel chamber and glue pockets thick layer of FIG. 7.

FIG. 9 schematically shows an example of a microfluidic chip connected to a reservoir.

In the drawings, identical reference numbers identify similar elements or components. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not necessarily intended to convey any information regarding the actual shape of the particular elements, and have been solely selected for ease of recognition in the drawings.

### DETAILED DESCRIPTION OF THE EMBODIMENTS

The examples presented herein are for the purpose of furthering an understanding of the invention. The examples are illustrative and the invention is not limited to the example embodiments.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one example” or “an example embodiment,” “one embodiment,” “an embodiment” or combinations and/or variations of these terms means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Definitions

Generally, as used herein, the following terms have the following meanings unless the context suggests otherwise:

As used herein, “BBB” is understood to mean blood-brain barrier, formed by brain specific vascular endothelium.

As used herein, “ELISA” has its generally accepted meaning and is understood to mean enzyme-linked immunosorbent assay.

As used herein, “HUVEC” has its generally accepted meaning and is understood to mean human umbilical vein endothelial cells.

As used herein, “PDMS” has its generally accepted meaning and is understood to mean polydimethylsiloxane.

As used herein, “plurality” is understood to mean more than one. For example, a plurality refers to at least 3, 4, 5, 70, 1,000, 10,000 or more.

As used herein, “TEM” is understood to mean tissue-engineered microenvironments.

As used herein, “RFP” is understood to mean red fluorescent protein.

As used herein, “vasculogenesis” is understood to mean the formation of syncytial, multicellular structures that have inner lumens.

As used herein, “quiescent’ is understood to mean a non-sprouting endothelial cell microvessel.

As used herein, “tissue” is defined as an ensemble of one or several similar types of cells from the same origin, together with extracellular matrix secretions, that is specialized to carry out one or more specific functions.

As used herein, “organ” means a higher level of organizational structure consisting of multiple tissues, where an organ function is only possible by the interaction of multiple tissues.

### Example Embodiments

Using elements of generating parent vessels from single suspensions of endothelial cells seeded into voids in extracellular matrix gels, the knowledge of how to make these parent vessels sprout in a desired direction, and the knowledge of creating tissue cords in matrix voids next to a parent vessel, we developed a system that can be used to vascularize small tissues, either in form of cell colonies or tissue fragments in microfluidic devices. The system is built on a microfluidic chip that, in its most basic form, has the ability to contain a three-dimensional hydrogel matrix with three voids of which at least two are all in fluidic connection with channels within the chip body. The fluid paths are separate to allow for establishing fluid pressure gradients between the endothelialized channels.

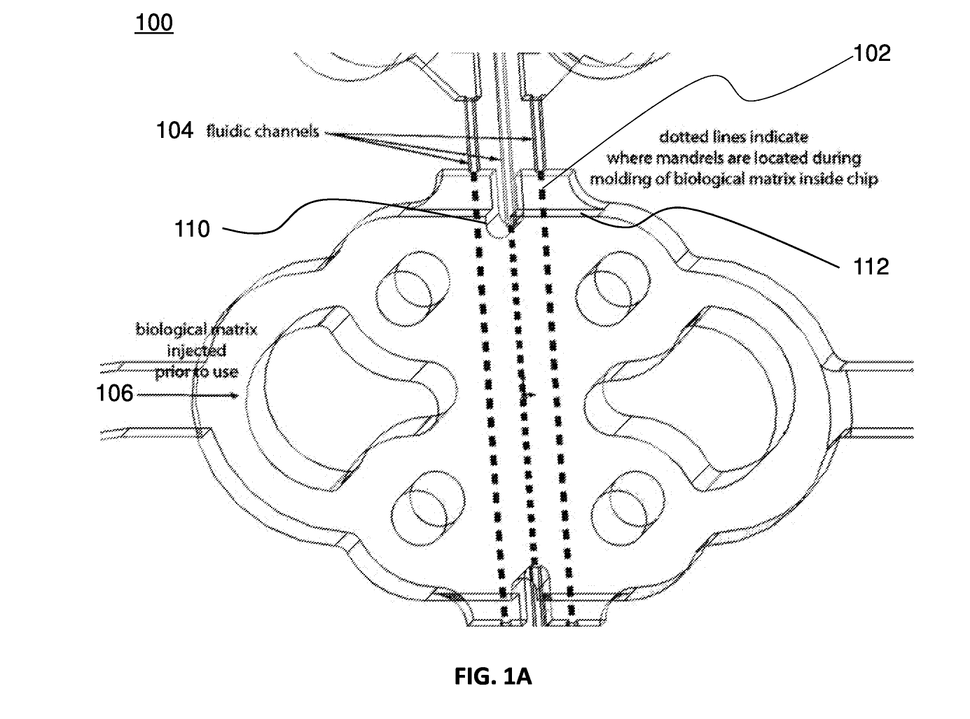

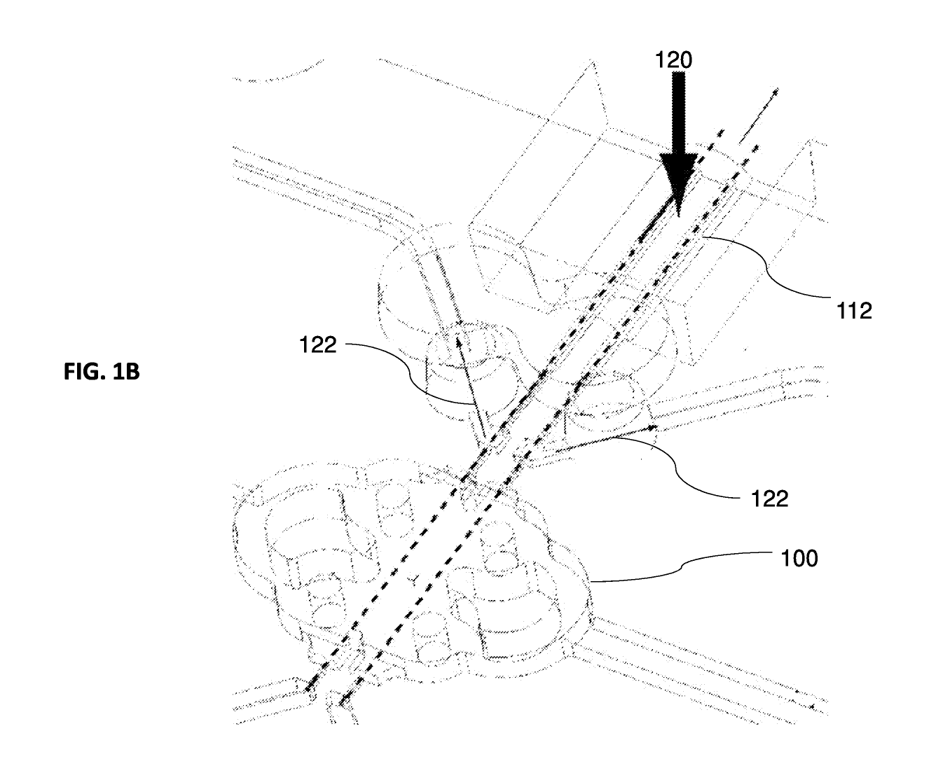

Referring now to FIG. 1A, an example of a 3-channel chip interior chamber, highlighting the fluidic channels and biological matrix chamber is schematically depicted. A 3-channel microfluidic cell culture chip 100 is made in a manner similar to that described in the publication (Tourovskaia et al. “Tissue-engineered microenvironment systems for modeling human vasculature” Exp Biol Med (Maywood) 2014 September: 239(9):1264-71). To make the chip 100 a plurality of mandrels (not shown), in this example three mandrels, may be inserted in the device to function as molding pins for the biological matrix. Three dotted lines 102 illustrate positions where inserted mandrels would exclude biological matrix, thus forming a plurality of fluidic channels (illustrated below) in biological matrix 106 that is connected with the fluidic channels 104 molded into the chip material. Additional barriers between the parallel channels (i.e. protrusions 110 from interior chamber wall 112) help maintain biological matrix connection to the chip wall, and reduce the possibility of fluidic shunting.

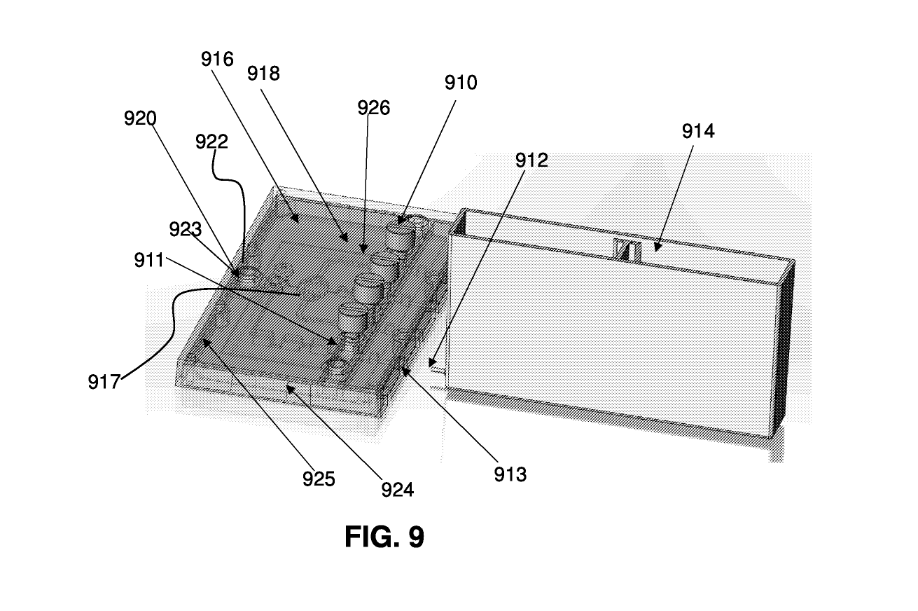

Referring now to FIG. 1B, an example of the application of a fluidic shutoff valve that enables rerouting of fluid through the mandrel channels is schematically shown. In one useful example, the distance between each of the plurality of channels may advantageously be less than 1 mm. As a result, since each mandrel must be pulled out of the chip without undergoing much bending if any, there is little space for robust connection of the chip to an external fluid source. In this example, connectors to the external fluid source can be spaced further apart. After biological matrix is injected and crosslinks, the mandrels are extracted from the chip through an on-chip shutoff valve indicated by arrow 120. The shut off valve may comprise a pinch off valve or the like. When the shutoff valve 120 is closed off fluid is re-routed via routing channels 122 to more conveniently-placed connectors (not shown) that are further apart. One example of such a shut off valve is illustrated in FIG. 9 below and described in more detail in PCT application no. PCT/US2015/056271, international filing date Oct. 19, 2015, entitled “MODULAR MICROFLUIDIC SYSTEM FOR PERFUSED CELL CULTURE,” to Neumann et al. which is incorporated herein by reference.

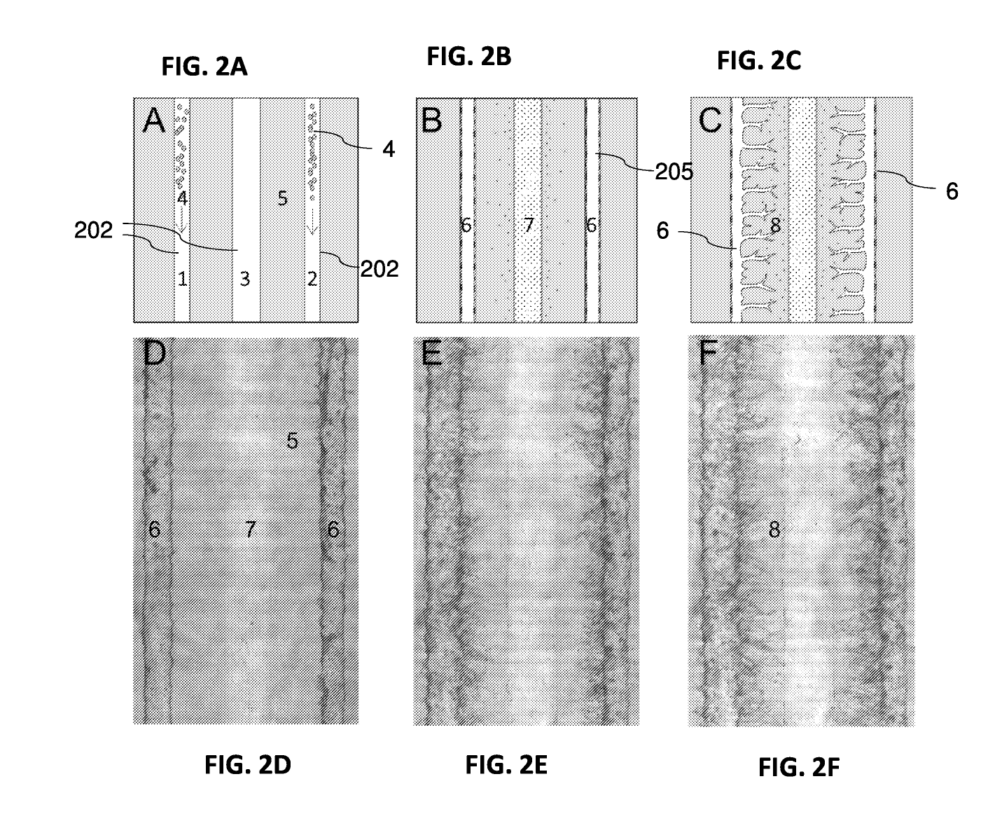

Referring now specifically to FIG. 2A, a schematic of a chamber in a microfluidic device in a three-channel setup is shown. Three voids 202 (e.g. channels) are created using mandrel extraction or degradation methods as described above. The chamber is filled with a 3D-matrix 5 (e.g. a collagen gel). The three channels 202 traverse the chamber and the left and right channels 1, 2 are endothelialized by infusing an endothelial-cell suspension 4. The central channel 3 is initially kept empty.

Referring now specifically to FIG. 2B, here endothelial cells 205 have attached to the inner surface of channels 1 and 2, resulting in two parent vessels 6 that are perfused with growth medium. Vascular growth factors 7 are infused into the central channel creating a growth factor gradient within the matrix.

Referring now specifically to FIG. 2C, angiogenesis is demonstrated. The parent vessels 6 respond to the growth factor gradient by directed angiogenesis toward the central channel as indicated by capillary vessels 8.

Now referring jointly to FIG. 2D-FIG. 2F an example of the establishment of two quiescent parent vessels in the outer channels that are then induced to sprout toward the center channel by perfusion of growth factors through the center channel are there demonstrated. The basic principle of vascularizing multicellular structures within the described devices is as follows. Suspensions of endothelial cells were infused in each of the outer two channels 6 (voids) within the matrix. (FIG. 2D). This was done by injecting endothelial-cell suspensions into the seeding ports in the device, as described by Tourovskaia et al. (Exp Biol Med (Maywood), 2014, 239). The cells traveled with the perfusate into the matrix chamber and quickly attached to the inner walls of the channels. Gels from Collagen-I are commonly used; however, a number of other gel types are suitable for cell seeding also. After the cells formed an endothelial lining inside the two outer channels resulting in two parent vessels (FIG. 2E), a vascular growth factor solution was introduced into the central channel, which induces the parent vessels on each side to sprout toward the central void (FIG. 2F).

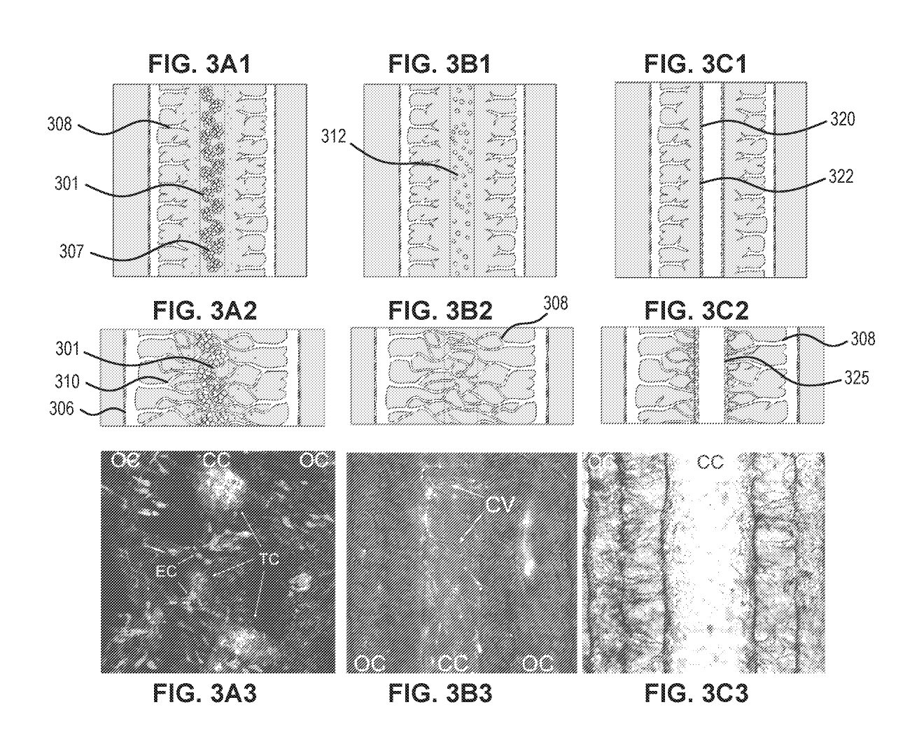

Now referring jointly to FIG. 3A1-FIG. 3C3 where vascularization of different tissue structures in a 3-channel chip is shown. More particularly in FIG. 3A3-FIG. 3C3, OC is an abbreviation for outer channel; CC is an abbreviation for center channel; EC is an abbreviation for endothelial cells; TC is an abbreviation for tumor clusters; and CV is an abbreviation for connecting vessels.

Now referring specifically to FIG. 3A1, multicellular structures 301, as for example, tissue fragments, biopsies, cancer cell colonies, stem-cell clusters, or pancreatic islets, are introduced into the central chamber 307 by perfusion/infusion. The cell structures attract the parent vessel sprouts 308 by secretion of vascular growth factors, which also can be supplemented by perfusion or by binding growth hormones to the matrix in the central channel Additional endothelial cells can be mixed in with the multicellular structures.

In FIG. 3A2, shown there is a microvascular network 310 that engulfs the cell fragments 301 and has connected with the sprouting parent vessels 306. In FIG. 3B1 a suspension of endothelial cells in a gelable matrix 312 (e.g. collagen) is introduced into the central channel. In FIG. 3B2 there endothelial cells responding to vascular growth factors supplemented into the gel undergo vasculogenesis and then connect with the angiogenic branches 308 from the parent vessels. In FIG. 3C1 adherent cells 320 form an epithelial or endothelial layer on the inner wall 322 of the central channel after being introduced as a cell suspension, allowed time to attach and wash out non-attached cells. Vascular growth factors can be added via lumenal perfusion of the central channel or by adding to the matrix. In FIG. 3C2 sprouts 308 from the parent vessels attach to the ablumenal side 325 of the cell tube.

In one example, when the sprouts have reached the central channel, the tissue of choice is added to the central channel. The tissue of choice can be tissue fragments collected from healthy or diseased tissues of humans or animals. For example, these tissues can be fragmented tumors, liver tissue, brain tissue, muscle tissue, adipose tissue, life tissue, dead tissue and so on. The tissues can be fragments of benign or cancerous lesions, biopsy material, tissues infected with microorganisms, or chemo-treated or radiated tissues. A number of tissue fractionation methods are well described in the literature. It is advantageous to select a fragment size that allows the fragments to be seeded into the devices without clogging or destroying the matrix channel in the chip. The tissues can be packed loosely or tightly into the central channel. The fragments can be also suspended into liquid matrix that will gel once the suspension is introduced into the channel. Endothelial cells can be mixed in between the fragments, enhancing the degree of vascularization of the tissue fragments once these endothelial cells undergo vasculogenesis and connect with the sprouts coming from the parent vessels on each side. The end result is a core of tissue in the central void that is vascularized by a microvascular network, with the microvascular network being in lumenal connection with a parent vessel on each side (FIG. 3A2). Growth medium, buffers, drugs and other substances can be then perfused through one parent vessel via the tissue core into the other parent vessel, basically mimicking a tissue unit with arterio-capillary-venous flow pattern.

This method can be used to create tissue models that investigate flow, tissue vascularization, oxygen consumption, as well as the interaction of the endothelium with certain tissue types. This is particularly of interest for cancer applications, the stimulation of insulin-producing cells, interactions between stem cells and endothelium. In a teratoma model, stem cell clusters, seeded in our chips in the presence of endothelial parent vessels formed all three germ layers, whereas stem-cell clusters seeded in non-endothelialized channels did not. There is a plethora of research questions that revolves around vascularized tissues. Also, for drug testing, test compounds can be subjected to the tissues via the fluid flow, which is physiologically more relevant than adding the compounds externally. The model would also be compatible with introduction infectious organisms via the fluid flow, for example malaria parasites (sporozoites of Plasmodium) into liver fragments. The system is also compatible with building a blood-brain-barrier system by having the central core populated by astrocytes, microglia and other brain specific elements, with a vascular network traversing the “brain core”.

A number of variations of the setup and cell and matrix elements comply with the described model. For example, the central core can be filled with a number of different tissue types. These can be fragmented tissue samples, either alone or in combination with fragments of other tissues. Other cells can be added to the tissue fragments, such as immune cells, macrophages, cancer cells, or infectious organisms. The tissues fragments can be embedded in the same matrix as used around the channels; it can be in different concentrations and stiffness's, it can be Matrigel, inert materials, specialized matrices, derived from specific tissues (e.g. lyophilized kidney or liver matrices). Alternatively, the fragments can be from decellularized extracellular matrix from special tissues or organs (leaving the structural, micro-architectural components of the extracellular matrix intact) that are then re-cellularized prior to seeding into the chip or after seeding into the chip. Besides using fragments of tissues and organs (biopsies, pieces from resected organs, isolated pancreatic islets) the method can also be used for cultured cells. These can be in the form of single cells, cell clusters, organoids, tumor spheroids, stem cell clusters, colonies of cardiomyocytes, or myogeneic cells, stem-cell derived pancreatic islets and others. The method is basically applicable to all kinds of live and dead tissues or even dead structures that are in contact with living tissues, such as foreign objects to model inflammation and scar formation, functionalized beads etc. Once again, these multicellular structures can be simply filled into the central chamber or mixed into a matrix material. And again, these multicellular structures can be mixed with endothelial cells that form vasculogenic structures that link with the two parent vessels and their sprouts.

Another structural variation of the method is to use the central channel to create tubular tissues. (FIG. 3C1). For example, kidney proximal tubule cells can be seeded into the central channel (as described in US PCT Application No. PCT/US2013/062307). The cells quickly form a tube that is then quickly vascularized from the sprouting parent vessels (FIG. 3C2). This method can be applied to all cell types that adhere to the matrix walls, in particular to replicating tubular organs, such as (but not limited to) intestine (See, for example, US PCT Application No. PCT/US2013/062307), seminiferous tubules, liver sinusoids, lymphatic vessels, blood vessels, a cardiac tube.

Another structural variation is filling the central channel with a gelable matrix material containing endothelial cells capable of vasculogenesis (FIG. 3B1). Endothelial cells in gels from collagen, fibrin, or others can, in response to appropriate stimulants, undergo vasculogenesis, which means they form syncytial, multicellular structures that have inner lumens. The networks of vascular structures will then connect with the branches from the parent vessels that were generated with angiogenic stimulants prior to filling the central channel (FIG. 3B2). The method combines angiogenesis followed by vasculogenesis. The method promises to be useful for generating perfused microvascular networks quickly and reliably. Additional cell types can be seeded in various concentrations to support vessel maturation and/or to mimic certain tissues, functions, or disease states (e.g. pericytes, astrocytes, stromal cells, cancer cells, microorganisms, viruses).

Another important variation of the method is the formation of entire cell cords in the central void. For example, cardiomyocytes or their progenitors can be packed into the central void either alone or in combination with endothelial cells. The cylindrical shape of the channel will orient the cardiomyocytes longitudinally; thus, their contraction will be aligned. The same can be achieved with skeletal muscle cells, smooth muscle cells or their precursors. As for the other mentioned variations of these method the protocols not only apply to healthy cells or tissues but also to diseased tissues. Other applications can be neuronal cords, bone, and bone marrow.

Another variation of the method is to seed the channels in a different temporal order and supply sprouting factors via a different channel. For instance, a kidney proximal tubule could first be established in the center channel, either alone or in combination with endothelial cells or other supporting cells. Then endothelial parent vessels could be created in one or both of the outer channels. Once complete non-sprouting parent vessel(s) (quiescent) are established, a first parent vessel can be induced to sprout toward the second parent vessel or channel by flowing sprouting media through the second outer channel/vessel. Such an approach would result in a vascular network initiated from one parent vessel, growing through the matrix and around a proximal kidney tubule (or other tissue) and connecting with a second parent vessel or channel.

A variation of this approach is to establish one or two quiescent parent vessels in the outer channels and to induce sprouting from one channel by flowing sprouting media through the second channel. Once a vascular network has been established, the center mandrel can be removed and the channel seeded with a variety of cells alone or in combination with gelled matrix and/or other cell types.

Another variation of this approach is to use a permeable hollow fiber, such as a cellulose fiber, as mandrel for the central channel. The permeable fiber can be perfused with vascular growth factors that permeate through the fiber wall into the matrix and attract sprout from the outer EC parent vessels. Once the parent vessel sprouts have formed a dense network around the hollow fiber, the fiber is removed and the remaining void filled with the tissue of choice.

Another variation is to establish a quiescent parent vessel in the center channel first and then induce it to sprout toward the outer channels by perfusing the outer channels with vascular growth factor enriched media. Once the sprouts are sufficiently close to the outer channels, induction of sprouting is stopped and cells can be seeded into one or both outer channels.

Another variation is to fill the center channel with endothelial cells dispersed in an extracellular matrix (either alone or in conjunction with other cell types or tissue components) and induce sprouting toward the outer channels by perfusing the outer channels with vascular growth factor enriched media until the sprouts have reached the lumens of the outer channels and endothelialized them.

### EXAMPLES

### Example 1: Directional Angiogenic Sprout Formation

Again referring jointly to FIG. 2B-FIG. 2F, after quiescent HUVEC parent vessels were established in the outer channels, the center mandrel was removed and the channel perfused with a cocktail of angiogenic growth factors (EGM-2 with VEGF, bFGF, and PMA: sprouting media;) as illustrated in FIG. 2B. Medium was refreshed every 3 days. This results in a continuous gradient of angiogenic factors across the matrix from the “source” channel (center) to the corresponding outer “sink” channels. As shown in FIG. 2C and FIG. 2D, FIG. 2E, and FIG. 2F within 4 days of exposure to sprouting media, sprouts 8 begin to form and grow toward the central channel, eventually spanning a distance>200 um. This method of initiating a vascular network in a 3-channel chip was used to create a variety of vascularized tissue structures.

### Example 2: Vascularization of Multicellular Structures

After the outer parent vessels were induced to sprout towards the center channel, the center channel was populated with human breast adenocarcinoma cells (MCF-7) mixed with HUVEC-RFP cells and collagen I. The cell suspensions were mixed in a ratio of 1:3 (MCF-7:HUVEC-RFP) in a 3 mg/ml collagen solution at a final cell concentration of 10×10⁶ cells/ml. The 3-channel chip was disconnected and the cell/collagen mix was injected into the center channel using a 1 ml syringe. The chip was left at room temperature for 15 minutes to allow cells to attach. The center channel ports and side ports were closed and perfusion was continued with standard endothelial cell media through the side channels for 24 hours. After 24 hours the center channel was perfused with sprouting media. The cultures were maintained for 7 days and then stained and imaged using confocal microscopy. As best shown in FIG. 3A3 the cultures were stained with antibodies specific for endothelial cells (PECAM), epithelial cells (EpCAM, breast cancer cells), and nuclei (DAPI). Endothelial cells seeded in the center channel were visualized using the RFP tag (red). By day 7, sprouts from the outer parent vessels OC had connected with microvessels MV formed in the center channel to support the tumor cell clusters TC.

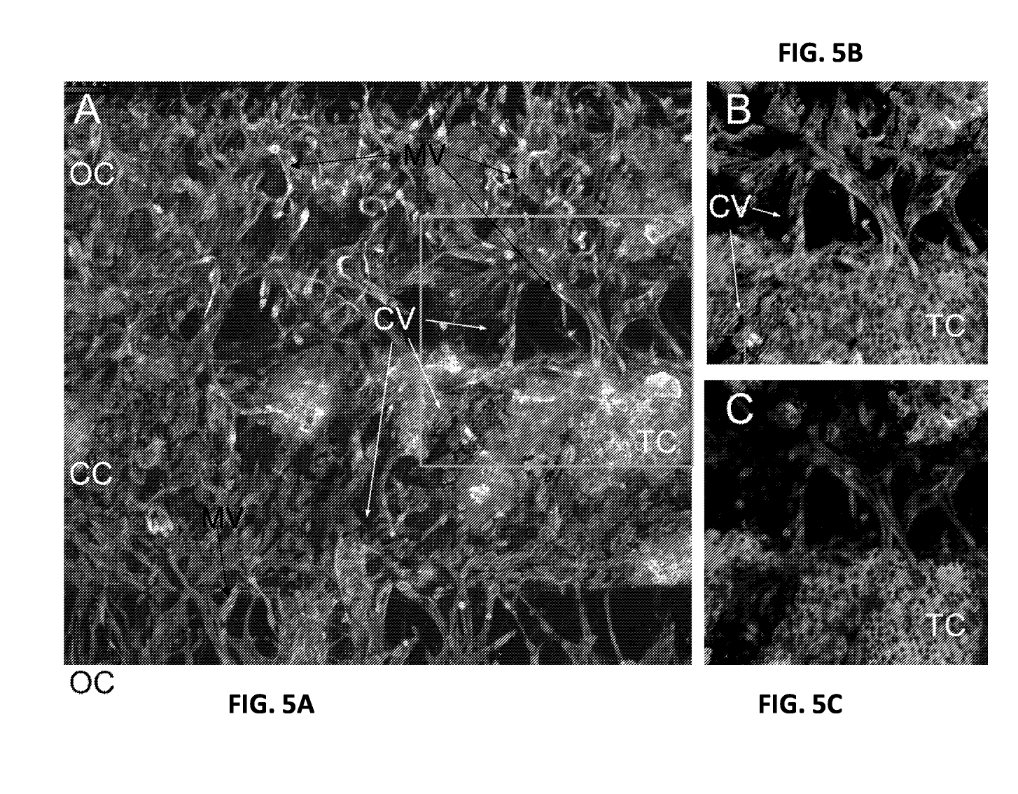

Referring to FIG. 5A-FIG. 5C vascularization of tumor cell clusters in the center channel and microvessels growing into and around tumor cell clusters are shown. Outer parent HUVEC vessels were induced to sprout toward the center channel CC, which was then populated with HUVEC-RFP cells and tumor cell clusters in collagen I. Sprouts from the outer channels connected with microvessels formed in the center channel CC (arrows). Sprouts from both parent vessels OC formed connections with the newly formed microvessels MV in the central channel (as shown in FIG. 5A). Further, these sprouts grew into and around the tumor cell clusters (TC; as shown in FIG. 5B and FIG. 5C).

### Example 3: Specific Tissue Application

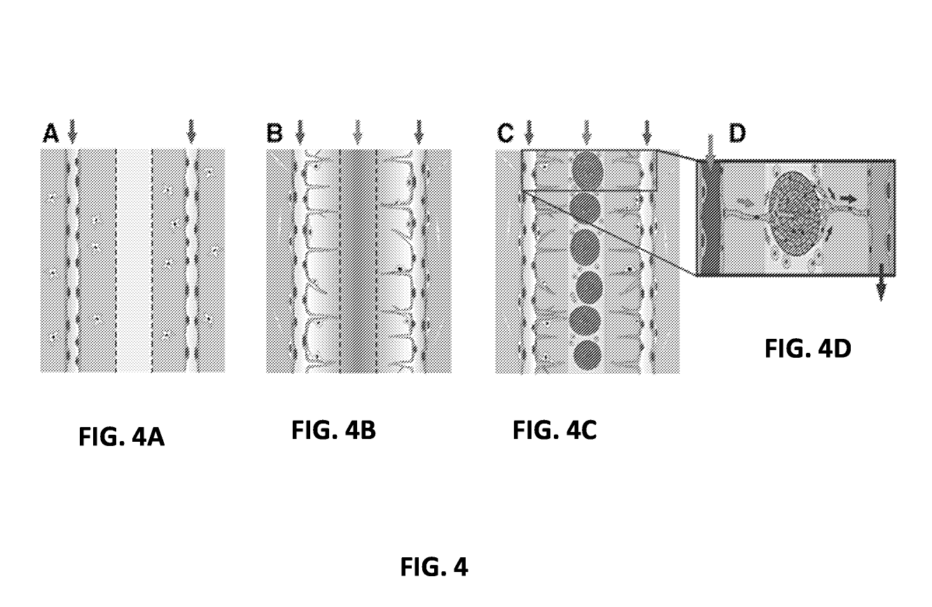

Described is a method designed for vascularizing pancreatic islets. The method is shown progressing from left to right in FIG. 4A-FIG. 4D. The system is designed to achieve a structure that resembles the human islet portal system, composed of an afferent arteriole leading into a sinusoidal capillary network that drains into an efferent venule outside the islet (FIG. 4D). The method uses a three-channel microfluidic chip. In order to fit human islets (either isolated from donors or created from stem cells), the central channel has a wider diameter (˜300 microns) in between the two vascular channels. This third channel enables to precisely place a zone of islets in between two sprouting blood vessels. The sequence of tissue-engineering steps is as follows: Using the mandrel approach, we first create three channels in a 3D collagen matrix. HUVECs are then seeded into the two outer channels; pericytes (support cells) will be embedded into the matrix (FIG. 4A). Similarly to the already established angiogenesis model (U.S. Pat. No. 7,622,298 B2), angiogenic sprouting from parent vessels will be induced toward the central channel by perfusing the latter with VEGF. (FIG. 4B). Once the capillary sprouts are close to reaching the central channel, we will inject pancreatic islets in collagen into the central channel (FIG. 4C). This will generate a zone of islets in between the sprouting vessels. VEGF-A produced by the islets will then induce the capillaries to grow into and around the islets. Once the two parent vessels are connected via the capillary network in the islet zone, a pressure difference will be generated between them, such as to re-route the perfusate from one parent vessel through the islet capillaries into the other parent vessel with the aim to replicate the islet blood-flow pattern in vivo (FIG. 4D).

Vascularization of Endothelial Cells in Gelable Matrix.

After inducing sprouting from parent vessels in the outer channels, red fluorescent protein (RFP)-expressing HUVECs (10×10⁶ cells/ml) were mixed with collagen I (3 mg/ml) and introduced into the central channel. The chip was left at room temperature for 20 minutes and then the center channel ports and matrix ports were closed and perfusion was continued through the outer channels for 24 hours. After 24 hours, the center channel was perfused with sprouting media. The cultures were maintained for 7 days and then stained and imaged. Within the center channel, the RFP-HUVECs underwent vasculogenesis and after 7 days of culture, these microvessels had formed connections with the pre-established sprouts from the outer parent vessels (FIG. 3B3, CV arrows).

Vascularization of a Cell Tube.

After inducing sprouting from parent vessels in the outer channels, HUVECs were seeded into the central channel. The chip was left at room temperature for 20 minutes to allow cells to attach and then the center channel ports and matrix ports were closed and perfusion was continued through the outer channels for 24 hours. After 24 hours, the center channel was perfused with sprouting media. The cultures were maintained for 7 days and then stained and imaged. Within 2 days of culture, HUVECs in the center channel had formed a complete tubular tissue structure. By 7 days, sprouts from the outer parent channels had attached to the ablumenal side of the central HUVEC vessel (FIG. 3C3).

Lumenal Connection within Established Vascular Network.

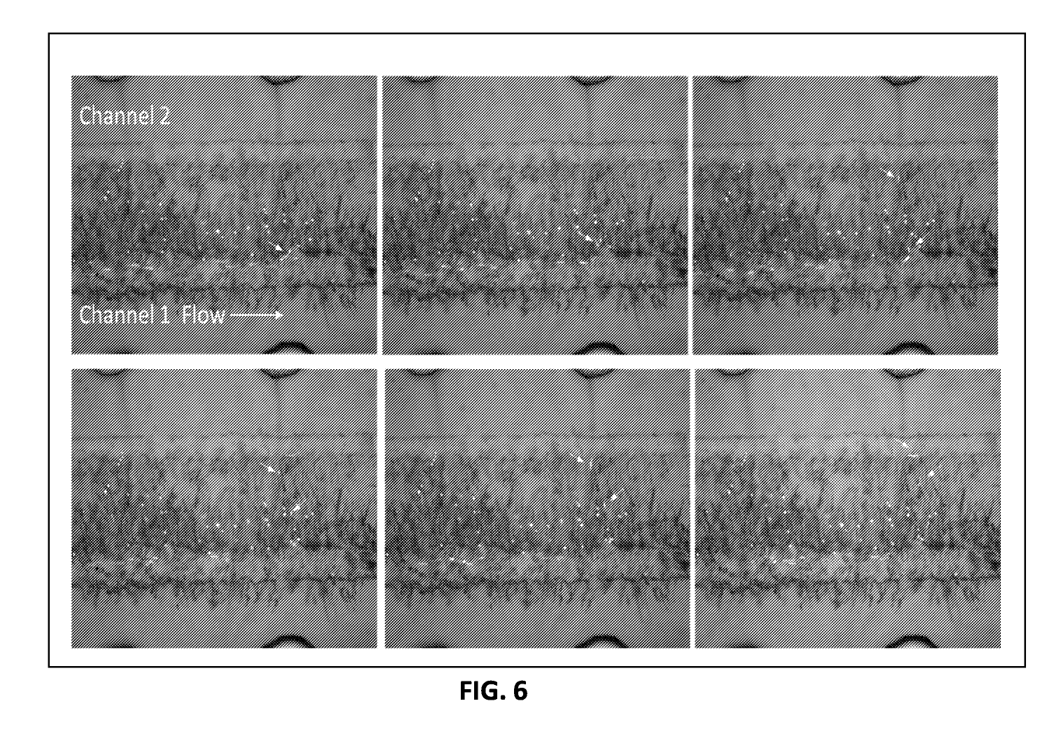

Two parent HUVEC vessels were established as previously described and induced to sprout with sprouting media (as described above). After a connecting vascular network had been established between the parent vessels, fluorescent beads (10 um) were perfused through one parent vessel and visualized in real time. Fluorescent beads were observed traveling from one parent vessel, through the connecting vasculature, and into the second parent vessel (as shown in FIG. 6). This flow pattern demonstrates lumenal connection within the established vascular network.

Generation of Outer Channel Parent Vessels.

Referring jointly to FIG. 2A, and FIG. 2D-FIG. 2F, collagen I (7 mg/ml) was injected into the culture chamber 5 of a 3-channel chip. After the matrix had gelled, the mandrels from the outer channels 1, 2 were removed and the chip fluidic pathways connected to a pneumatic perfusion platform. Standard endothelial cell culture media was used to prime the side channels prior to cell seeding. The chip's matrix side ports were closed while the channel ports remained open and regulated incubator pumps were used to pneumatically drive fluid flow through the chip. HUVEC cells were harvested at a concentration of 10×10⁶ cells/ml and a cell suspension 4 (2.5 ul) was injected into each of the side channels using a 22 gauge non-coring needle in a 2.5 ul Hamilton syringe. After injection the chips were left untouched for 15 minutes and the matrix side ports were closed and the outer channels opened. The platforms were returned to the incubators and perfusion reinstated. Endothelial cell channels were perfused with media to allow formation of complete non-sprouting (quiescent) endothelial cell parent vessels 6 in the outer channels (as best shown in FIG. 2D).

Any of the described methods can be based on cell lines, primary cells, stem cells, cells from healthy or diseased donors, and combinations thereof. Currently, there is an increased need for tissue-model systems that include a vascular and perfusion component for scientific research and drug development. The described models could serve as important tool to study a number of important diseases, such as cancer, cardiovascular disease, diabetes, inflammation, aging, and neurodegenerative diseases.

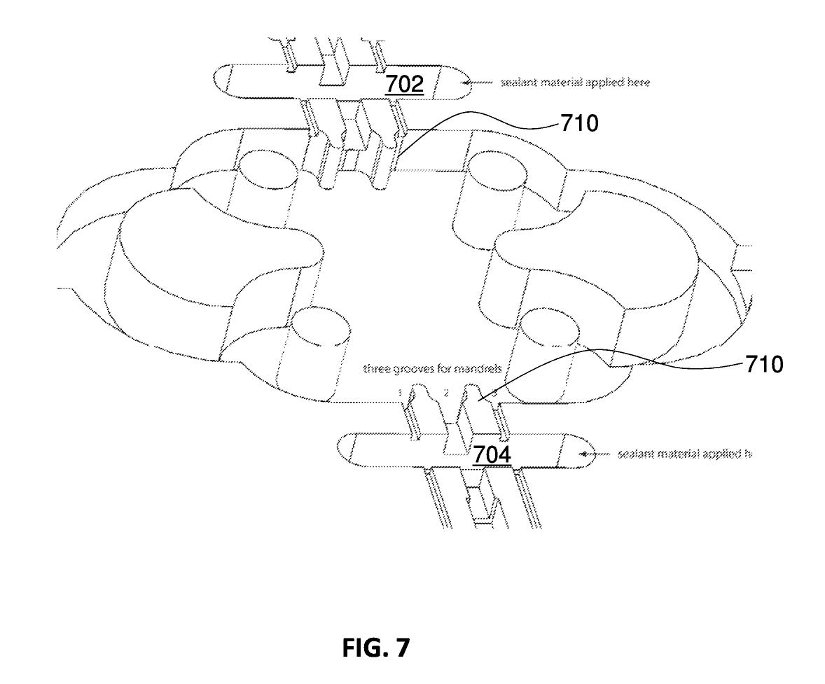

Referring now to FIG. 7 an example of a 3 channel chamber and glue pockets thick layer is illustrated. In order to seal mandrels into a chip in close proximity sealant is applied in regions 702 and 704. The sealant, typically PDMS silicone, is applied to create a form fitting seal of each mandrel to the chip. The adhesion of the sealant to the chip is maximized while the adhesion of the sealant to the mandrel is minimized. In end use, after extracellular matrix (i.e. collagen) is injected and gels inside the chip, the mandrel should be extracted with minimal disturbance to the system.

Due to the small size of these features, the design of the pocket in the chip that accommodates mandrel sealant specifies that all three mandrels are sealed in one operation. Means of sealing mandrels into chip provides seamless transition between chip material and extracellular matrix. The geometry of each channel 1, 2, 3 that accommodates a mandrel is designed to align and center each mandrel. The addition of sealant means that the channel formed between the chip material (i.e. silicone) and the extracellular matrix is circular in cross-section and identical in size on both the chip material and extracellular matrix sides. This tight seal prevents leakage of the ungelled extracellular matrix mixture when injected into the main chamber. Leakage of the injected extracellular matrix into the lumenal perfusion channels is avoided. Chemical means of modifying the surface of the chip allows bonding of the extracellular matrix to the chip wall. This provides more controlled fluidics without creating a shunt path for fluid along the extracellular matrix-chip boundary.

The purpose of this barrier is to provide extra surface area and path length that helps to prevent delamination of the extracellular matrix from the chip wall. Delamination of the extracellular matrix from the chip wall produces shunt paths between channels and results in a loss of control of fluidics and loss of independence of perfusion of one or more channels. Independent perfusion of the extracellular matrix channels enables fluid to be routed through anastomosed blood vessels, with a center channel consisting of an organ cell mass or cell tubule.

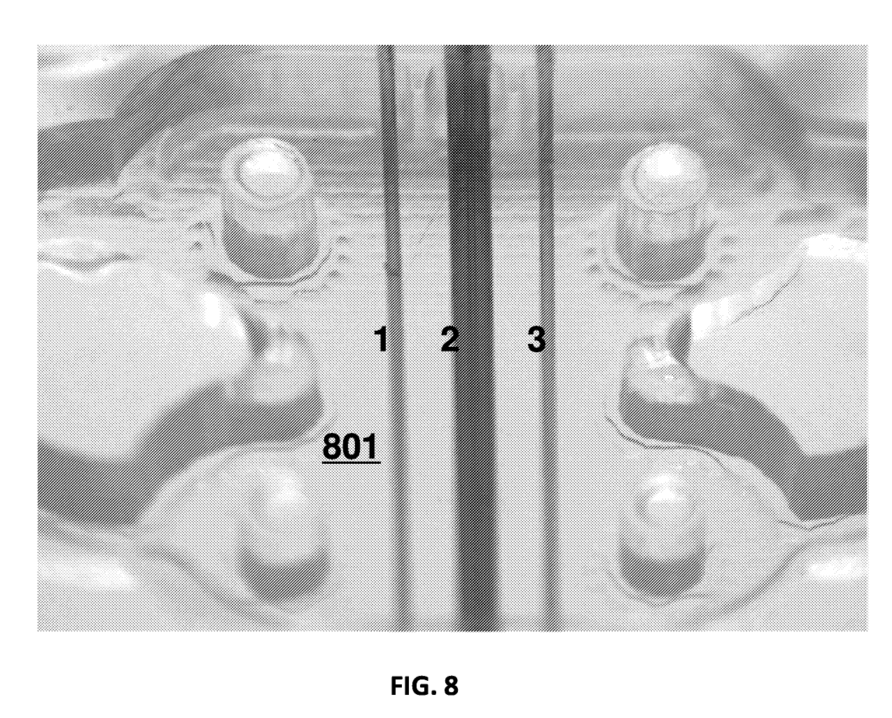

Referring now to FIG. 8, a more detailed view of the 3 channel chamber is shown. The extracellular matrix channels 1, 2 and 3 are located in the matrix chamber 801 after withdrawal of previously inserted mandrels.

Referring now to FIG. 9, an example of a microfluidic chip connected to a reservoir is shown. A microfluidic chip 916 includes a microfluidic circuit 918. The microfluidic circuit 918 includes a biological chamber 917 coupled to a plurality of fluid channels 926 onto which are mounted a series of shut-off valves 911, where each shut off valve 911 includes a valve actuator 910. A reservoir 914 includes a fluidic connector 912 sized to couple to one of an array of connectors 913. In one example, the chip 916 consists of a thin, flat bottom side and an upper side with a high-aspect ratio thin walled channel that is depressed by the actuator.

In one example, the chip 916 contains a cylindrical channel 20 with defined dimensions and a protruding ring 922 sized to fit into a cylindrical hole 923 in an upper chip shell 924. This feature creates a compression seal over a straight-shaft connector of a defined outer diameter (OD). The chip 916 contains a linear array of the connectors 913 at defined intervals to allow connection to a syringe, a pipette tip, a medial reservoir, a collection reservoir or to another chip through a jumper tube (not shown). The flow path is set by the placement of media (source) and collection reservoirs to given channels on the chip and by user actuated shut-off valves 911 located on the chip, allowing multiple flow path possibilities. The number of channels 926 is scalable to allow the design to work with chips with different flow configurations. It also creates a more stable system due to the elimination of tubing runs and connections between the chip 916 and reservoir 914.

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by specifically different equipment, and devices and reconstruction algorithms, and that various modifications, both as to the equipment details and operating procedures, may be accomplished without departing from the true spirit and scope of the present invention.

1. A method for vascularizing cell aggregates or tissue segments in a microfluidic device comprising:
filling a chamber within the microfluidic device with a matrix that allows for endothelial sprouting;
creating at least three voids within the matrix, of which at least two outer voids are lumenally connected to separate perfusion paths within the device and at least a one inner void is positioned in between the at least two outer voids;
endothelializing the at least two outer voids;
introducing at least one cell type, cell aggregation, matrix material, tissue segment, or combinations thereof into the void between the two outer voids; and
using vascular growth factors to induce the endothelial cells to sprout into the matrix until the at least three voids are interconnected by endothelial sprouts.

2. The method of claim 1 in which the sequence of populating the at least three voids with cells comprises following steps:
first, endothelializing the at least two outer voids by lumenal seeding of endothelial cells;
second, introducing angiogenic factors into the at least one inner void in between the two endothelialized voids, causing the two endothelialized voids to grow endothelial sprouts toward the at least one inner void in;
third, filling the at least one inner void with cell aggregates or tissue fragments; and
fourth, letting the endothelial sprouts form vascular connections that extend from one endothelialized void through the at least one inner void containing cell aggregates or tissue fragments to the second endothelialized void, with the result of having created a vascularized tissue in the chip.

3. The method of claim 1 wherein the sequence of populating the at least three voids with cells comprises following steps:
first, filling the at least one inner void that is in between at least two outer voids which are lumenally connected to at least two perfusion paths within the device with endothelial cells, matrix materials, cell aggregates, tissue fragments, or combinations thereof;
second, introducing angiogenic factors into the at least two outer voids, causing the endothelial cells to grow endothelial sprouts towards the at least two outer voids;
third, letting the endothelial sprouts invade and endothelialize the at least two outer voids, and having formed a vascular network that extends from the at least one inner void to the at least two outer voids.

4. The method of claim 1 wherein the at least three voids are created by using removable mandrels and applying following sequence of steps:
positioning at least three mandrels in parallel within the matrix such that there are at least two outer mandrels and at least one inner mandrel between the two outer mandrels;
after the matrix is gelled, removing the mandrels from at least two outer positions, generating the at least two outer voids;
endothelializing at least one of the two outer voids by lumenal seeding with endothelial cells and perfusing the endothelialized at least one other outer void with an angiogenic factor inducing the endothelialized channel to grow endothelial sprouts toward the outer void that is perfused with angiogenic factors;
removing the at least one inner mandrel from the matrix when the endothelial sprouts have formed a connection between the at least two outer voids and are grown around the inner mandrel; and
filling cells, cell aggregates, tissues, matrix materials or combinations thereof into the channel that is created by removing the at least one inner mandrel.

5. The method of claim 1 wherein the at least one inner void is generated by using a removable permeable fiber that is perfused with vascular growth factors until a network of endothelial sprouts has grown from the at least two outer endothelialized voids connecting around the removable permeable fiber; and after removal of the permeable fiber, filling the resulting channel with endothelial cells, matrix materials, cell aggregates, tissue fragments, or combinations thereof.

6. The method of claim 1 wherein the at least one inner void is perfused with materials selected from the group consisting of nutrient solutions, test substances, blood, blood components, blood surrogates, and cells in solution.

7. The method of claim 1 claims wherein the microfluidic device is fabricated from a polymer selected from the group consisting of a polymeric organosilicon compound, silicone, polydimethylsiloxane (PDMS), cyclic olefin copolymer, polystyrene, and polycarbonate.

8. The method of claim 1 wherein the chamber and paths are embedded in a substrate juxtaposed between a glass plate and a polycarbonate, or rigid clear thermoplastic, plate.

9. The method of claim 1 wherein the matrix is selected from the group consisting of gelled synthetic or naturally occurring hydrogels, Collagen I, fibrin, combinations of Collagen I, IV, hyaluronan, chitin, chitosan, alginate, agarose, gelatin, synthetic matrices, biologically inspired synthetic (hybrid) matrices, and combinations thereof.

10. The method of claim 1 wherein the cells populating the at least one inner void are derived from the group comprised of pancreatic islet cells, endothelial cells, kidney cells, intestinal cells, liver cells, cardiac cells, skeletal muscle cells, smooth muscle cells, lung cells, testis cells, kidney cells, cancer cells, immune cells, mammalian cell lines, invertebrate cell lines, primary cells, stem cells, single cell suspensions, microorganisms, parasites, cell aggregates and tissue fragments.

11. The method of claim 1 wherein sprouting from the endothelialized voids is induced by vascular growth factors perfused through the at least one void that is positioned in between the at least two outer voids.

12. The method of claim 1 wherein the sprouting from the endothelialized outer voids is induced by factors released by cells or tissues placed into the at least one void that is positioned in between the at least two outer voids.

13. The method of claim 1 wherein the sprouting from the endothelial cells is induced by growth factors released from the matrix in the at least one inner void.

14. The method of claim 1 further comprising forming a cellular tube in the at least one inner void by seeding cells that attach to the walls of the inner void and form an endothelial or epithelial cell sheet.

15. The method of claim 14 further comprising perfusing the cellular tube with fluid.

16. The method of claim 14 further comprising perfusing the cellular tube with gas.

17. The method of claim 1 further comprising introducing single cells, cell clusters, or tissue fragments into the at least one inner void.

18. The method of claim 1 further comprising suspending single cells, clusters of tissue fragments or combinations thereof into a liquid matrix that is subsequently gelled within the at least one inner void.

19. The method of claim 18, wherein the single cells include endothelial cells, further comprising inducing the endothelial cells to undergo vasculogenesis forming vascular networks that form a connection between the at least two endothelialized outer voids and the at least one inner void.

20. The method of claim 1 further comprising creating a pressure difference between the two outer endothelialized voids and routing perfusate from the endothelialized outer void with the higher pressure through the vascular sprouts around or through the at least one inner void into the other endothelialized outer void with the lower pressure.

21. (canceled)
